SEQUENCE LISTING (11 sequences)

SEQ ID NO: 1 - DNA, 30 bp, Artificial Sequence
  Random primer
ggactgacgaattccagtgactnnnnnnnn

SEQ ID NO: 2 - DNA, 30 bp, Artificial Sequence; identical to SEQ ID NO: 1
  Random primer

SEQ ID NO: 3 - DNA, 22 bp, Artificial Sequence
  Primer; Tag-ONLY
ggactgacgaattccagtgact

SEQ ID NO: 4 - DNA, 17 bp, Artificial Sequence
  primer
gcctgtactgggtctct

SEQ ID NO: 5 - DNA, 21 bp, Artificial Sequence
  primer
aagcactcaaggcaagcttta

SEQ ID NO: 6 - DNA, 21 bp, Artificial Sequence
  Primer
tgttagagtggaggtttgaca

SEQ ID NO: 7 - DNA, 20 bp, Artificial Sequence
  Primer
caatcyggactgagacctgc

SEQ ID NO: 8 - DNA, 20 bp, Artificial Sequence
  Primer
acgctgtaaacgatgcacac

SEQ ID NO: 9 - DNA, 20 bp, Artificial Sequence
  Primer
gacgtcatcctcaccttcct

SEQ ID NO: 10 - DNA, 17 bp, Artificial Sequence
  Primer
gcctgtactgggtctct

SEQ ID NO: 11 - DNA, 21 bp, Artificial Sequence
  Primer
aagcactcaaggcaagcttta

(19) United States
(12) Patent Application Publication
(10) Pub. No.: US 2012/0024701 A1
(43) Pub. Date: Feb. 2, 2012

(54) GENERAL PROCEDURE FOR THE IDENTIFICATION OF DNA SEQUENCES GENERATING ELECTROMAGNETIC SIGNALS IN BIOLOGICAL FLUIDS AND TISSUES

(75) Inventors: LUC MONTAGNIER, New York, NY (US); Claude Lavallee, Lexington, MA (US); Jamal Aissa, Chatillon (FR)

(73) Assignee: Luc Montagnier, New York, NY (US)

(21) Appl. No.: 13/168,367

(22) Filed: Jun. 24, 2011

Related U.S. Application Data
(60) Provisional application No. 61/476,545, filed on Apr. 18, 2011.
(60) Provisional application No. 61/476,110, filed on Apr. 15, 2011.
(60) Provisional application No. 61/358,282, filed on Jun. 24, 2010.

(51) Int. Cl. G01N 33/559; G01R 27/04; C07H 21/00; C09K 3/00; B82Y 15/00

(52) U.S. Cl. 204/456; 536/23.1; 252/408.1; 324/629; 250/493.1; 977/700

(57) ABSTRACT

A general method for producing EMS positive samples or samples containing nanostructures characteristic of self-replicating molecules like DNA by dilution and agitation. Methods of transduction into DNA information or for inducing EMS in an originating sample and transducing the EMS signal once induced into a naïve receiving sample. Diagnostic methods using this technology.

### CROSS-REFERENCE TO RELATED APPLICATIONS

This application claims priority under 35 U.S.C. §119(e) to U.S. 61/358,282, filed Jun. 24, 2010; U.S. 61/476,110, filed Apr. 15, 2011, and U.S. 61/476,545, filed Apr. 18, 2011. Each of these documents is incorporated by reference in its entirety.

### STATEMENT REGARDING FEDERALLY SPONSORED RESEARCH OR DEVELOPMENT

(not applicable)

### REFERENCE TO MATERIAL ON COMPACT DISK

(not applicable)

### BACKGROUND OF THE INVENTION

1. Field of the Invention

Induction, detection and transmission of electromagnetic signals (EMS) from self-replicating molecules like DNA. Transduction of EMS from an EMS positive (EMS+) sample to a naïve, unsignalized sample. Methods for identifying a molecule like DNA in a sample by transducing its EMS signature to water, amplifying the signalized water to produce a DNA. Methods for detecting DNA associated with a condition, disorder or disease of incomplete or unknown etiology by inducing specific EMS emission from the sample at a particular frequency, signalizing a naïve sample with the emitted EMS, and detecting an EMS in the signalized water and/or amplifying the signalized water using a DNA amplification technique and analyzing the products of the amplification.

2. Description of the Related Art

The inventors have previously described a method for selectively detecting DNA sequences of pathogenic microorganisms by their emission of low frequency electromagnetic waves (EMS) in water dilutions. U.S. application Ser. No. 12/560,772, filed Sep. 16, 2009, entitled “System and Method for the Analysis of DNA sequences in Biological Fluids” discloses a method for detecting electromagnetic waves derived from bacterial DNA, comprising extracting and purifying nucleic acids from a sample; diluting the extracted purified nucleic acids in an aqueous solvent; measuring a low frequency electromagnetic emission over time from the diluted extracted purified nucleic acids in an aqueous solvent; performing a signal analysis of the low frequency electromagnetic emission over time; and producing an output, based on the signal analysis, in dependence on the DNA in the sample. The products and procedures as well as other subject matter disclosed in this patent application are expressly incorporated by reference.

Methods for detecting some low electromagnetic frequency electromagnetic signals in diluted filtrates of the culture medium of certain bacteria and viruses, as well as in diluted plasma of patients infected by the same agents are disclosed by U.S. application Ser. No. 12/097,204, PCT/FR2007/001042, filed Jun. 22, 2007, and U.S. application Ser. No. 12/797,826, filed Jun. 10, 2010 each of which expressly incorporated by reference in their entirety. The electromagnetic signals (EMS) were believed to be produced by certain defined nanostructures induced by the microorganism, in high dilutions in water, after the microorganism had been removed by filtration.

Materials and methods for detecting replicating molecules such as DNA and methods for EMS detection as well as other subject matter pertinent to the present invention disclosed in these documents is incorporated by reference to the following documents:

U.S. Pat. No. 6,541,978, WO 00/17638 A (Digibio; Benveniste, Jacques; Guillonnet, Didier) 30 Mar. 2000 (2000-03-30).

U.S. Ser. No. 09/787,781, WO 00/17637 A (Digibio; Benveniste, Jacques; Guillonnet, Didier) 30 Mar. 2000 (2000-03-30);

U.S. Ser. No. 09/720,634, WO 00/01412 A (Digibio; Benveniste, Jacques; Guillonnet, Didier) 13 Jan. 2000 (2000-01-13);

FR 2,811,591 A (Digibio) 18 Jan. 2002 (2002-01-18);

FR 2,700,628 A (Benveniste Jacques) 22 Jul. 1994 (1994-07-22).

Benveniste J. et al: “Remote Detection Of Bacteria Using An Electromagnetic/Digital Procedure”, Faseb Journal, Fed. Of American Soc. For Experimental Biology, Bethesda, Md., US, No. 5, Part 2, 15 Mar. 1999 (1999-03-15), page A852, XP008059562 ISSN: 0892-6638.

Thomas et al: “Activation Of Human Neutrophils By Electronically Transmitted Phorbol-Myristate Acetate” Medical Hypotheses, Eden Press, Penrith, US, vol. 54, no. 1, January 2000 (2000-01), pages 33-39, XP008002247, ISSN: 0306-9877;

Benveniste J. et al.: “Qed And Digital Biology” Rivista Di Biologia, Universita Degli Studi, Perugia, IT, vol. 97, no. 1, January 2004 (2004-01), pages 169-172, XP008059428 ISSN: 0035-6050;

Benveniste J. et al.: “A Simple And Fast Method For In Vivo Demonstration Of Electromagnetic Molecular Signaling (EMS) Via High Dilution Or Computer Recording” FASEB Journal, Fed. Of American Soc. For Experimental Biology, Bethesda, Md., US, vol. 13, no. 4, Part 1, 12 Mar. 1999 (1999-03-12), page A163, Abstr. No. 016209, XP008037356 ISSN: 0892-6638;

Benveniste J: “Biological effects of high dilutions and electromagnetic transmission of molecular signal” [Progress In Neonatology; 25th National Conference Of Neonatology] S. Karger Ag, P.O. Box, Allschwilerstrasse 10, CH-4009 Basel, Switzerland; S. Karger Ag, New York, N.Y., USA Series: Progres En Neonatologie (ISSN 0251-5601), 1995, pages 4-12, XP009070841; and 25ES Journees Nationales De Neonatologie; Paris, France; May 26-27, 1995 ISSN: 3-8055-6208-X;

Benveniste et al.: “Abstract 2392” FASEB Journal, Fed. Of American Soc. For Experimental Biology, Bethesda, Md., US, 22 Apr. 1998 (1998-04-22), page A412, XP009070843 ISSN: 0892-6638;

Benveniste et al.: “Abstract 2304” FASEB Journal, Fed. Of American Soc. For Experimental Biology, Bethesda, Md., US, 28 Apr. 1994 (1994-04-28), page A398, XP009070844 ISSN: 0892-6638; and

U.S. Pat. Nos. 7,412,340, 7,081,747, 6,995,558, and 6,952,652.

In some instances, it was demonstrated that the EMS could originate from specific genes or even from some fragmented DNA sequences. This was discovered to be the case for the adhesin gene of Mycoplasma pirum (U.S. Ser. No. 12/097,204, filed Dec. 14, 2006) and of the LTR (Long terminal repeat), nef and pol genes of Human Immunodeficiency Virus (HIV) (U.S. 61/186,610, filed Jun. 12, 2009 & U.S. Ser. No. 12/797,826, filed Jun. 10, 2010). However, for many microbial agents or diseases of unknown origin or etiology this identification was not possible. Consequently, the inventor developed new methods, disclosed herein for detecting and identifying biological molecules, specifically DNA or other nucleic acids, associated with these other disease or disorders.

### BRIEF SUMMARY OF THE INVENTION

There are several nonlimiting aspects to the invention.

(1) A method for producing a solution, such an aqueous solution like water that contains nanostructures that characterize a molecule like DNA. This method involves dilution, usually serial dilution, of a sample containing DNA and agitation of the sample between dilutions to produce the water nanostructures.

(2) Measuring EMS characteristic of a molecule like DNA or of its nanostructure in an originating sample and transducing this signal into a second receiving sample, usually water that does not emit the EMS signal. This is performed without contacting the originating sample and the receiving sample.

(3) Electronic transmission of a detected or recorded EMS signal to a remove location and optionally imprinting it on a naïve sample and/or recovering DNA or other replicating molecule from the imprinted naïve sample.

(4) Detecting DNA or DNA like molecules in a sample suspected of containing a particular agent, like HIV or Borellia.

(5) Identifying DNA or similar molecules present in an unknown sample, such as from a sample from a subject having a disease of unknown etiology.

(6) Devices that detect, induce, transduce or transmit EMS signals.

### BRIEF DESCRIPTION OF THE DRAWINGS

FIG. 1 illustration of apparatus and method for EMS signal transduction. Tube 1 contains a sample of DNA dilution positive for EMS. Tube 2 initially contains unsignalized or naïve water. After exposure inside coil to 7 Hz excitation signal, naïve sample converts and emits EMS when diluted up to D4 (10⁻⁴). D-4 LTR HIV DNA (104 bp) 7 Hz, 18 Hrs and then PCR (35 cycles) from D2 to D15 after filtration 450 and 20 nM; DW: Distilled Water; FD2: Dilution 10⁻² after filtration at 450 nM and 20 nM.

FIG. 2 Detection by PCR of HIV1 LTR transduction in water.

FIG. 2A: HIV1 LTR DNA D6 (EMS positive) dilution was used as emitter using excitation frequency of 7 Hz during 18 hours in the apparatus described in FIG. 1 and placed close to the water receiver Tube 2. Like the latter, it was then diluted at 10⁻², refiltered by 450 nM and 20 nM filters and diluted to 10⁻¹⁵. Each dilution was then amplified by PCR 35 cycles. Note the DNA bands detected at dilutions D2, (FD2), D3, D4, and D5.

FIG. 2B shows transmission in water of D6 dilution of LTR HIV DNA (104 bp). Method was performed using excitation frequency 7 Hz, an 18 hr exposure followed by 35 cycles of PCR from D-2 to D-15 after 450 nM and 20 nM filtration. DW denotes distilled water control. FD2-FD15, dilution to 10⁻²-10⁻¹⁵. Transmission in water of D-4 LTR HIV DNA (104 bp) 7 Hz, 18 Hrs and then PCR (35 cycles) from D-2 to D-15 after filtration 450 and 20 nM. Note: DNA band formation is up to D-8.

FIG. 3. Illustration of method to generally identify an unknown DNA sample. DNA in plasma sample is induced to emit EMS and the EMS signal is transduced to a separate sample of water to produce signalized water. Water signalized by EMS is serially diluted and PCR is performed using random tag primers producing DNA. The sequence of the DNA is determined and can be compared to known DNA sequences to identify the DNA in the unknown sample. Example 3 describes such a method.

FIG. 4. Detection of unknown DNA sequences from a patient plasma DNA sample. DNA was extracted from the plasma of a patient suffering from chronic Lyme disease. A D9 (10⁻⁹) EMS positive dilution of the original DNA sample was transduced into water by excitation at 7 Hz for 18 hrs. PCR was performed on dilutions of the receiving water sample. FIG. 4 shows agarose gel electrophoresis of the transduced DNA obtained after PCR with Tag8N primers followed by a second PCR with the Tag primers only. Three DNA bands were observed. As shown at the left, results obtained when the tube of D9 DNA and the tube of water are placed side-by-side. At right, results obtained when the two tubes were placed at a distance of 4 cm from each other during the 7 Hz excitation. Dw denotes control, naïve, unsignalized water. Dw vor: denotes control naïve, unsignalized water agitated with a vortex. D0: water that was transduced but not diluted. D2 NF: same as D0 but diluted by 1:100 (D2). D2 same as D2 NF, but filtered. D3, D4, D5 represent further serial dilutions of D2 to factors of 1:1,000 (D3); 1:10,000 (D4) and 1:100,000 (D5). All serial dilutions were vortexed between each 1:10 dilution.

### DETAILED DESCRIPTION OF THE INVENTION

Definitions:

Nucleic acid: Includes single stranded, double stranded DNA, and RNA as well as modified polynucleotide sequences. Biological samples containing DNA associated with a disease or disorder are generally isolated or recovered in double stranded form.

Self-replicating molecule: A molecule, such as DNA, that under appropriate conditions, can reproduce the information content of its primary, second, tertiary or quaternary structure. For example, a DNA molecule can replicate itself in the presence of the appropriate enzymes, primers and nucleotides.

DNA Amplification: Methods for amplifying nucleic acids are known. Conventional methods including polymerase chain reaction (PCR) are known and are also incorporated by reference to Current Protocols in Molecular Biology (updated Apr. 5, 2010), Print ISSN: 1934-3639; Online ISSN: 1934-3647.

Nanostructures: These structures of water are induced by biological molecules like nucleic acids such as single stranded or double stranded DNA. While not being bound to any particular theory, according to the physical theory of diphasic water, filtration and mechanical agitation (succussion) are believed to induce in water a low energy potential favoring the formation of quantum coherent domains. These domains will become replicas of a DNA molecule and vibrate by resonance when properly diluted and excited; see Del Guidice, et al., Water as a Free Electric Dipole Laser, Phys. Rev. Lett. 61, 1085-1088 (1988). Hydrogen bonding networks in liquid water, such as those described by Cowan, et al., Nature 434 (7030): 199-202 (2005) have not been associated with nanostructures.

Serial Dilutions: Serial dilution is a well-known technique and involves the stepwise dilution of a substance, such as DNA, in a solvent, such as water, saline solution, aqueous buffer, or an aqueous alcohol solution. Generally, serial dilutions as performed herein are stepwise dilutions by a factor of 10, or dilution of 1 part of a more concentrated solution in 9 parts of a solvent.

EMS: Electromagnetic signal. EMS in the context of the methods herein generally involves those having frequencies ranging from 0 Hz to 20,000 Hz as well as all intermediate subranges and values. Components of the ambient electromagnetic field include Schumann resonances which represent a set of spectrum peaks in the extremely low frequency (ELF) portion of the Earth's electromagnetic field spectrum. Schumann resonances are global electromagnetic resonances excited by lightning discharges in the cavity formed by the Earth's surface and the ionosphere and are the principal background in the electromagnetic spectrum between 3 and 69 Hz. A representative Schumann resonance peak occurs in the Earth's electromagnetic spectrum and an ELF of about 7.83 Hz. By comparison, 60 Hz cycling of electricity is used in North America and 50 Hz elsewhere in the world.

EMS detection. Any suitable means for interrogating a sample and measuring its EMS may be employed. Exemplary systems, methods, and apparatuses for this purpose are disclosed by Butters, et al., WO 03/083439 A2, and are incorporated by reference to this document. Generally, these procedures will involve placing a sample into a container having electromagnetic and magnetic shielding, a source of Gaussian noise for injection in to the sample, a detector for detecting an electromagnetic time-domain signal composed of sample source radiation superimposed on the injected Gaussian noise, and a storage device for storing the time-domain signal and a time-domain signal separately detected from the same of a similar sample.

EMS Signature: The EMS characteristic of a particular biological molecule or a time domain signal associated with a material of interest. EMS signatures for various biological molecules are disclosed by U.S. Ser. No. 12/797,826, filed Jun. 10, 2010. Such EMS signatures as well as methods for producing samples suitable for EMS detection and methods for detecting an EMS signature are incorporated by reference to this patent application.

An EMS Signature of a particular molecule can be represented by a characteristic electromagnetic time domain signal. An EMS Signature may be recorded and replayed, undergo signal transformation or processing, or be transmitted.

Excitation Frequency: A frequency used to excite a sample in which an EMS signature has been detected and induce an EMS signature in a sample previously devoid of the EMS signature, e.g., pure water. These frequencies include those of 7 Hz or above, e.g., 7, 8, 9, 10, 11, 12, 13, 14, 15, 20, 30, 40, 45, 50, 55, 60, 65, 70 or more.

Originating Sample: A biological sample that contains an EMS signature, such as one characteristic of one or more biomolecules. An example would be a sample containing an EMS signature characteristic of DNA derived from human immunodeficiency virus.

Receiving or Signalized Sample: A sample, such as water or another aqueous buffer or dipole that has acquired or been imprinted with a nanostructure corresponding to a biological molecule, such as DNA. Methods for producing signalized water by serial dilution and agitation in water or in an aqueous solvent are disclosed herein.

Pathogenic Disease: Disease caused by or associated with a pathogen, such as a pathogenic parasite, yeast or fungus, bacterium, virus or infectious protein, such as a prion. Examples include parasitic diseases such as malaria or trypanosomiasis, fungal diseases, such as infections caused by or associated with Aspergillus, Candida, Histoplasma, Pneumocystis, Cryptococcus, Stachybotrys (black mold), bacterial infections such as Lyme Disease, sexually transmitted bacterial infections, tuberculosis, viral infections, including HIV infection, herpes virus infection, or hepatitis, and prion associated diseases such as Creutzfeldt-Jakob disease and so-called Mad Cow disease.

Autoimmune Disease, Degenerative Disease, Disorders or Conditions: These diseases, disorders or conditions may or may not have been previously associated with a particular biological molecule, such as a particular DNA molecule or its corresponding water nanostructure. Examples include allergic conditions, multiple sclerosis, rheumatoid arthritis, disorders associated with transplantation or replacement of body parts, Alzheimer's disease, Parkinson's disease and other diseases or disorders of unknown or incomplete etiology, such as Chronic Fatigue Syndrome, Gulf War Syndrome, or with exposure to particular biological, chemical or physical agents or with the sequela of such exposure.

Representative embodiments of the invention are described below.

(i) Originating and Signalized Samples.

Test samples used to produce an EMS will contain DNA or other replicating biological molecules that can form nanostructures or can be naïve samples signalized by EMS transduction to emit EMS or contain nanostructures representative of the DNA or other molecule. Representative test samples include blood, plasma, serum, CSF, joint fluid, saliva, mucous, semen, vaginal fluid, sweat, urine, and feces. Tissue samples and samples from other sources, including laboratory or hospital sources, foods, drinks and potable water may be used. These may be diagnostic samples, such as those obtained from a subject known to have or suspected of having a particular conditions, disorder or disease like AIDS or Lyme disease. Alternatively, they may be obtained from subjects having or suspected of having a condition, disorder or disease of unknown etiology, such as a parasitic or fungal disease or disorder, bacterial disease or disorder viral disease or disorder, an autoimmune disease, disorder or condition, diseases such as Alzheimer's Disease or Parkinson's Disease.

To produce a sample that emits detectable EMS, a test sample undergoes dilution, usually serial dilution, and agitation preferably between each serial dilution. A test sample is usually diluted by a factor of 10³, 10⁴, 10⁵, 10⁶, 10⁷, 10⁸, 10⁹ 10¹⁰, 10¹¹, 10¹², 10¹³ or more. Though any intervening factor of dilution or other degrees of dilution that produce detectable EMS may also be used. The beginning concentration of a nucleic acid in a sample prior to dilution generally ranges from 1 ng/ml to 4 ng/ml.

Solutions for dilution and agitation as well as for containing an originating or receiving sample are preferably water, but other aqueous or dipolar solutions may be employed so long as they can provide nanostructures representative of DNA or other replicating molecules or induce detectable EMS when used. Examples of solutions include water, or other aqueous solutions, such as normal saline, phosphate buffered saline, physiologically acceptable aqueous solutions, buffered aqueous solutions, or alcohol and water mixtures, including 10, 20, 30, 40, 50, 60 and 70% or more of ethanol or other alcohol solutions or other solvents selected on a basis of their relevant properties depending on the molecule to be tested, may be employed in the methods described herein.

In some applications, control samples are required. The type of control sample may be selected by one of skill in the art depending on the particular application but in general will not emit the EMS signature of the molecule of interest or contain nanostructures corresponding to it. Often, such controls will constitute pure, unsignalized water, distilled water or pyrolyzed water or other solutions known to be nucleic acid free.

Signalized samples or solutions producing an EMS signature should not be boiled, heated or frozen for long periods of time so as to preserve the EMS signatures or nanostructures they contain. Preferably, these samples or solutions should be stored above freezing and less than 40° C.

Various forms and time periods for agitation are contemplated and are incorporated by reference to the documents mentioned above. Vortexing for a period of 15 seconds between serial dilutions is one representative method for producing a sample emitting detectable EMS.

(ii) EMS Transduction. The invention also relates to a method for producing an EMS signature in an aqueous buffer comprising placing an originating (EMS+) sample in an aqueous buffer and a receiving sample not having the EMS signature next to each other inside of an electromagnetically shielded container, applying an electromagnetic field for a time and under conditions sufficient to transfer the EMS signature from the originating sample to the receiving sample. The electromagnetic field is generally applied by a coil, such as a copper coil, located inside of an electromagnetically shielded container. Coils made of other electrically conducting metals or alloys may be employed or other devices that produce similar electromagnetic flux. The electromagnetic field can be applied to the sample for a time period ranging sufficient to produce an EMS signature, for example, from 12 to 24 hrs although other suitable time periods may be selected based on the nature of the sample, the sample dilution and the physical characteristics of the apparatus. Exposure time is chosen based on the amount of time required for transfer to occur. Some representative times include >0, 1, 2, 3, 4, 4-8, 8-12, 12-18, 18-24 and 24-48 hrs or longer. Signalized samples produced by this method as well as nucleic acids like DNA amplified from a signalized sample are also contemplated. Alternatively, an EMS signature may be imprinted in water or another aqueous buffer by contacting the one or more receiving samples with a recorded or transmitted and optionally amplified EMS signature previously obtained from an originating sample in an aqueous buffer having an EMS signature, for a time and under conditions sufficient to imprint the recorded or transmitted EMS signature of the originating sample onto the one or more receiving samples. Imprinting may be performed using means for applying an electromagnetic field, for example using a device, such as a copper coil or solenoid coil, optionally located inside of an electromagnetically shielded container. The electromagnetic field is applied to the sample for a time period sufficient to produce an EMS signature in the sample, for example for a period of 1 to 24 hrs. Other suitable time periods may be selected based on the nature of the sample, the sample dilution and the physical characteristics of the device or other means for applying the electromagnetic field. Signalized samples produced by this method as well as nucleic acids like DNA amplified from a signalized sample are also contemplated.

(iii) EMS Recording/Transmission. EMS signals once measured may be recorded on a tangible medium, such as a computer hard drive, a flash drive, DVD, or CD or other known media. They may be transmitted electronically, for example, over the internet, or by any other means that preserves the signal integrity. Recorded or received signals can be amplified and used to transduce EMS into a naïve solution as described above. This aspect of the invention can involve the recording, transducing, storing, and/or transmission of an EMS signature of a nucleic acid, such as that produced after serial dilution of a signalized sample. An EMS signature may be recorded by a suitable electronic device, such as a recorder, computer or computer network. The recorded EMS signature may undergo signal processing or signal transformation for example into a digital or analog signal, be transmitted by a communications device, such as via radio, telephone, modem, or Internet transmission to a receiver, such as a receiving computer, anywhere in the world.

A stored or transmitted EMS signature is then reconstituted and/or amplified and contacted with a receiving sample to imprint it with the EMS signature and produce nanostructures in the water or dipole solution of the receiving sample. Such a signal may be amplified prior to or after transmission, for example, using a commercial amplifier (e.g., Conrad). The electrical output from the amplifier containing the EMS signature is then applied to an electrically conducting coil (e.g., of copper wire) as described herein in which a plastic tube of pure non-signalized water or other dipole solution has been inserted for a time sufficient for imprinting of the EMS signature, generally for a period of at least one hour.

The production of EMS is then verified in water dilutions of the signalized water or dipole solution. The positive dilutions can be used for retrieving the DNA by PCR as described above. The DNA is then amplified by cloning and its sequence determined to be 98-100% identical to the initial DNA. This development will be useful for remote diagnosis or use in other telemedicine procedures or protocols.

The inventors previously discovered that an electromagnetic signal of low frequency (EMS) induced in a water dilution by the DNA of some kinds of bacteria and viruses can be transmitted at a distance into a naive or unsignalized water, aqueous medium or other dipole solution. It has also been discovered that such an EMS corresponding to a particular biomolecule like DNA (i.e., an EMS signature of a particular molecule), can be recorded. This involves recording EMS from DNA fragments obtained by PCR (polymerase chain reaction) with sequence specific primers in an electromagnetic coil. The resulting amplified current is connected to a computer and stored as a file, such as an analog or digital file (e.g., a digital sound file). The recorded EMS can then undergo signal processing, for example a digital sound file can be processed using computer software for storage, transmission, or use.

DNA may be reconstituted from its EMS signature. For example, the recorded or remotely transmitted EMS signature of a DNA molecule is input into a soundcard and the output from the soundcard is linked to an amplifier. Amplifier output is connected to a transducer solenoid into which an unsignalized water sample is placed. After a certain time, depending on the type of EMS signature, its intensity and the exposure time, the unsignalized water becomes signalized. In other words, the unsignalized water has memorized the EMS signature of the originating DNA molecule. By use of PCR the originating DNA molecule may be retrieved from the water signalized with its EMS signature. Verification of retrieval of the originating DNA sequence from the signalized water or verification of the fidelity of its reproduction can be verified by DNA sequencing.

Alternatively, prior to retrieval and synthesis of the DNA molecule by PCR, the signalization of the receiving sample with a DNA EMS signature may be determined by detecting the EMS emissions of the signalized sample using dilutions of the signalized water as previously described, e.g., by the device used to record the originating DNA sample's EMS signature in the first place. Only EMS positive dilutions will yield the DNA sequence. The procedure allows the transmission of DNA EMS signatures of medical interest as well as the remote retrieval of the corresponding originating DNA. Such transmission may be made by a medium of choice, for example, a digital signal may be transmitted over the internet or by sending USB keys (e.g., flashdrives) to remote laboratories or medical units.

(iv) Detection of a Known Nucleic Acid Sequence. Specific molecules known or suspected to be contained in a test sample may be screened using the methods described above. A test sample is diluted and agitated to produce an EMS+ sample and a nucleic acid amplification using specific known primers for the nucleic acid sequence of interest is performed. The test sample may be a sample produced by dilution and agitation or may be produced by tranduction of EMS into a naïve sample. An EMS+ test sample is incubated with primers for a specific nucleic acid sequence and the nucleic acid product by PCR amplification, usually DNA, is recovered. The recovered amplification products may be assayed indicate the presence of the particular nucleic acid in the test sample.

(v) Identification of an Unknown Nucleic Acid.

Another embodiment of the invention involves detecting a nucleic acid or nanostructures associated with an unknown nucleic acid in a test sample comprising amplifying a nucleic acid in a test sample using random nucleotide sequence or polynucleotides or primers; diluting and agitating during dilution the amplified nucleic acids in an aqueous solvent; measuring over time a low frequency electromagnetic emission from the diluted amplified nucleic acids; and optionally (i) identifying an EMS signature for amplified nucleic acid or its associated nanostructures by comparing the EMS of the test sample to the EMS of a control sample, and optionally (ii) comparing the results to relevant standard EMS signature(s). This method may further comprise performing a signal analysis of the low frequency electromagnetic emission over time, and/or producing an output, based on the signal analysis. This method may detect a biological molecule, such as a nucleic acid like DNA in a test sample and/or may detect a nanostructure derived from or associated with a nucleic acid such as DNA in the test sample. A suitable dilution of the test sample is selected for use within this method, for example, the test sample can be diluted by a factor of at least 10⁴, 10⁵, 10⁶, 10⁷, 10⁸, or 10⁹.

The test sample will usually be obtained from subject suffering from or at risk of developing a particular disease, disorder or condition. For example, the test sample can be obtained from a subject having or suspected of having a parasitic or fungal disease or disorder, a subject having or suspected of having a bacterial disease or disorder, a subject having or suspected of having viral disease or disorder, from a subject having or suspected of having had an autoimmune disorder, a subject having or suspected of having Alzheimer's Disease or Parkinson's Disease or any other neurological disease, a subject having or suspected to have a genetic disease or a gene alteration, or a subject having a disease, disorder or condition of unknown or incomplete etiology in comparison with a noninfected subject. For instance, an EMS signature of an HIV gene sequence, such as that of nef or pol, may be detected in a sample in comparison to a sample not containing the HIV gene sequence. Verification of the presence of a gene sequence in a sample may be made by PCR.

(vi) Devices. Various devices for use in conjunction with the different aspects of the invention are also disclosed. These include:

A device for producing an EMS signature in an aqueous buffer comprising at least two containers, at least one for an EMS originating sample and at least one for an EMS receiving sample, an electrically conducting coil that can emit a variable frequency ranging from 1 to 20,000 Hz, optionally connected to an external generator of alternating current having a variable frequency from 1 to 20,000 Hz, means for electromagnetic shielding the at least two containers and the electrically conducting coil.

A device or other means for transmitting at a distance EMS emitted by a biological sample or by nanostructures contained in a sample is also contemplated. Such a device will contain at least two containers, at least one to contain a sample determined to produce EMS characteristic of a DNA or a similar molecule in a first tube (originating sample), and another tube (receiving sample) to receive emitted EMS and contain signalized water produced. The device will contain an electrically conducting coil linked to an external generator of alternating current having a variable frequency from 1 to 20,000 Hz. The device will have shielding means, such as mu metal ≧1 mm in thickness, capable of isolating external ambient electromagnetic signals or noise, enclosing a space into which will accommodate the coil and the containers. Any suitable material may be used to make the coil and the elements and design of the coil are selected based on the size of the samples, shielding, and other elements of the apparatus. One example of a coil is a copper coil with the following characteristics: bobbin with internal diameter 50 mm, length 80 mm, R=3.6 ohms, 3 layers of 112 turns of copper wire, field on the axis to the centre 44 Oe/A, and on the edge 25 Oe/A. An example of shielding is a cylinder of μ metal having a minimal thickness of 1 mm, closed at both ends in a manner that completely isolates the enclosed containers and coil from the external ambient electromagnetic noise.

The following Examples describe particular embodiments of the invention, but the invention is not limited to what is described in these Examples.

### EXAMPLE 1

### Production of Samples Containing an EMS Signature Characteristic of HIV DNA

Step A:

Synthesis of DNA by PCR

A particular DNA sequence is first synthesized by polymerase chain reaction (PCR) on a DNA template, for example, a region of the LTR sequence present in the viral DNA extracted from the plasma of a HIV infected patient or obtained from a purified infectious DNA clone of HIV1 Lai, is amplified by PCR and nested PCR with respectively LTR-derived outer and inner primers.

Those were chosen to pick up some conserved regions of the LTR, given to several subtypes of HIV1. This amplified DNA was sequenced and found 100% identical to the known sequence of the prototype strain of HIV1 subtype B, HIV1 LAI (3). The resulting amplicon was determined to be 488 bp long and the nested-PCR amplicon to be 104 bp long.

Filtration and Dilution: A sample of each amplicon is prepared at a concentration of 2 ng/ml in a final volume of 1 ml of pure water that had been previously filtered through a sterile 450 nM Millipore (Millex) filter and then to a 20 nM filter (Whatman, Anotop) to eliminate any contamination by viruses or bacteria. All manipulations are done under sterile atmosphere in a biological safety cabinet.

The DNA solution is diluted one in 100 (10⁻²) in 2 ml of water and filtered through a 450 nM Millex filter (Millipore) and filtered again through an Anotop filter of porosity size 20 nM (Whatman).

The resulting DNA filtrate (there is practically no DNA loss through filtration, as the DNA molecules do not bind to the filters), is then diluted serially 1 in 10 (0.1 ml in 0.9 ml of water in an Eppendorf sterile tube of 2 ml from 10⁻² to 10⁻¹⁵.

A strong vortex agitation was performed at each dilution step for 15 seconds.

Each dilution in its stoppered plastic tube was placed on a coil under the ambient electromagnetic background at room temperature for 6 seconds; the resulting electric current is amplified 500 times and analyzed in a Sony laptop computer with specific software as previously described. The EMS positive vibrating dilutions (usually between 10⁻⁴ to 10⁻⁸) were detected not only by new peaks of frequency, but also quantitatively by the difference in amplitude and intensity of the signals measured in the software, as compared to the same parameters given by the background noise.

Table 1 shows the role of excitation frequency in inducing EMS from DNA into water. A fragment of LTR DNA (Tar region, 104 base pairs) was amplified by PCR with specific primers from the entire genomic HIV1 LAI DNA cloned in a plasmid (pLAI2). The fragment was purified by electrophoresis on an agarose gel; the DNA band was then cut and extracted with a Qiagen kit. Time of exposure DNA tube and water tube to the exciting frequency was 18 hrs.

TABLE 1

| | | | | Positive |
|---|---|---|---|---|
| Content | Frequency (Hz) | EMS | % over noise | dilutions |
| LTR DNA 104 bp | 2 | + | 33.3 | D6→ D8 |
| Water | | − | 1.2 | |
| DNA | 3 | + | 39.6 | D4→ D7 |
| Water | | − | 0.5 | |
| DNA | 4 | + | 43.9 | D5→ D8 |
| Water | | − | 1.5 | |
| DNA | 5 | + | 41.6 | D5→ D8 |
| Water | | − | 0 | |
| DNA | 6 | + | 33.5 | D5→ D8 |
| Water | | − | 1 | |
| DNA | 7 | + | 40 | D6→ D8 |
| Water | | + | 43.9 | D5→ D8 |

Step B:

Producing a Signalized Sample from the Originating Sample

Tube 1 containing one of the dilutions found positive for EMS in step A (10⁻⁶) was placed in the vicinity of an identical tube 2 that had been previously filled with 1 ml of pure water under a separate safety cabinet different from the one utilized in step A for the DNA manipulation. Both tubes were placed inside a copper coil with the following characteristics: bobin with internal diameter 50 mm, length 80 mm, R=3.6 ohms, 3 layers of 112 turns of copper wire, field on the axis to the centre 44 Oe/A, and on the edge 25 Oe/A, linked to an external generator of alternate electric current of variable frequency from 1 to 20,000 Hz.

The tubes and the coil were enclosed in a cylinder of thick (1 mm) μmetal closed at both ends in order to isolate the system from the external ambient electromagnetic noise. A current intensity of 100 mA was applied to the coil, so that no significant heat was generated inside the cylinder.

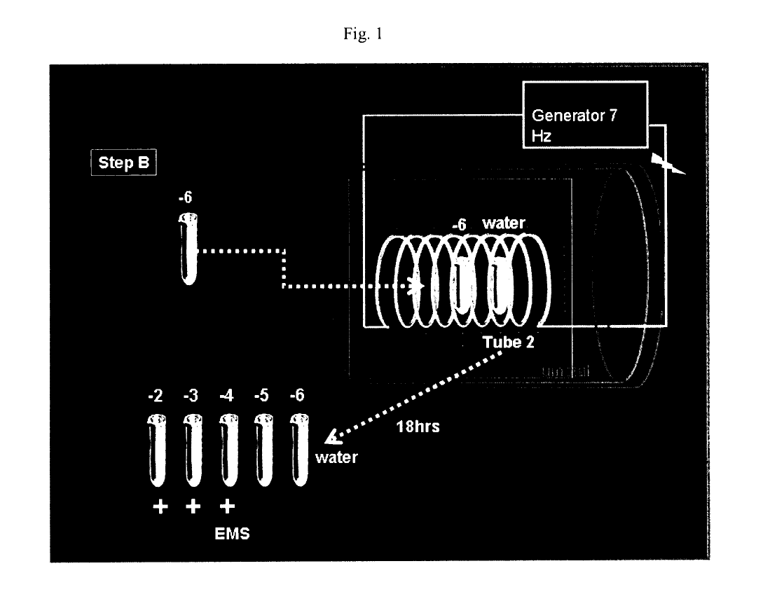

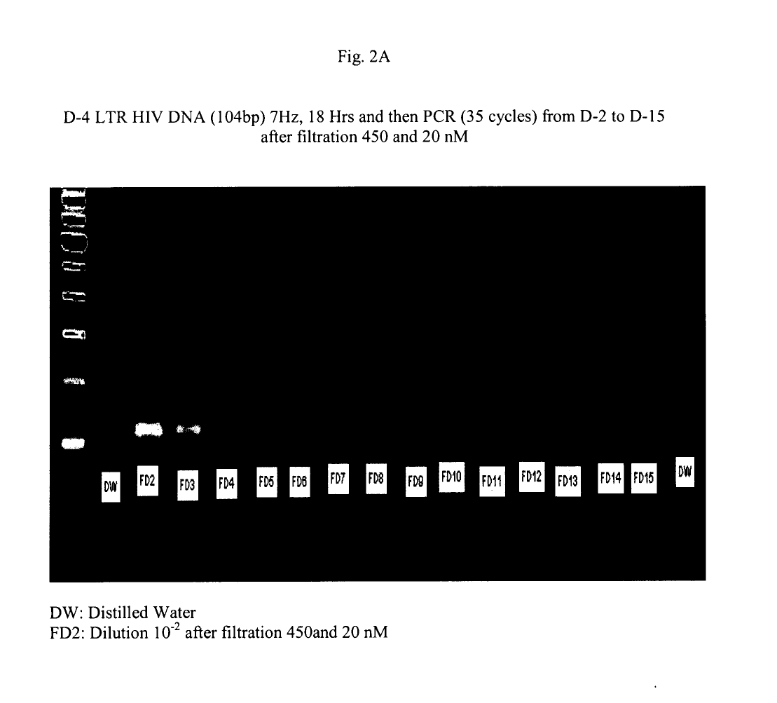

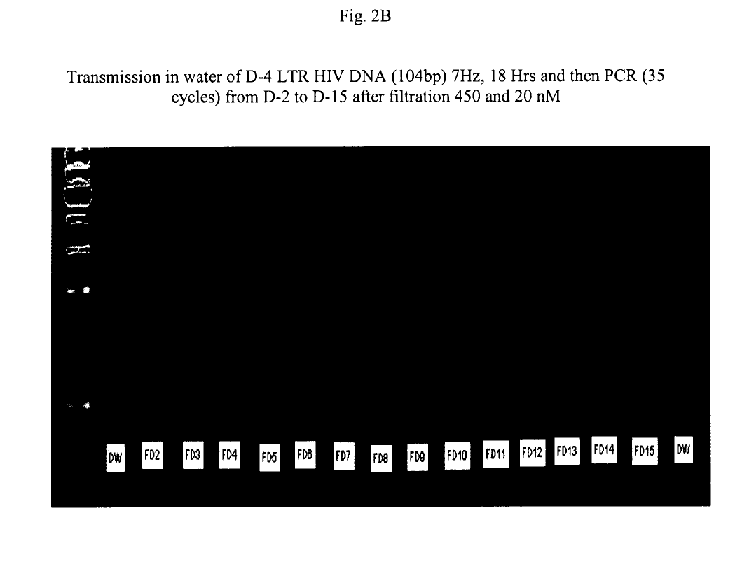

The tubes were kept 18 Hrs at room temperature in an oscillating magnetic field strength of 25 Oe/A generated by the coil system. Afterwards, the signalized water of tube 2 is filtered on 450 nM and 20 nM filters and diluted from 10⁻² to 10⁻¹⁵. As a control, the tube 1 was also filtered and diluted in the same way. EMS analysis revealed positive dilutions for EMS, starting at 10⁻² which is explained if one takes into account that the emitter tube 1 was already at the 10⁻⁶ dilution (FIG. 1). As shown in Table 1 a minimal frequency of 7 Hz was found necessary and sufficient to induce the EMS in the naïve, unsignalized water filled tube 2. However, the intensity of the EMS signals was sometimes reduced by comparison to those found in tube 1. To determine conditions suitable for EMS transduction, the inventors also varied different parameters of the process. It was determined that the following conditions suppressed EMS emission from naïve tube 2 (receiving sample or sample to be signalized).

Time of exposure of the two tubes less than 16-18 hrs (Table 2).

No coil.

Generator of magnetic field turned off.

Frequency of excitation<6 Hz.

No use of DNA in tube 1.

Tube 2 frozen at −80° C. overnight and defrosted before recording the EMS.

Tube 2 heated at 95° C. for 60 minutes after the overnight exposure.

Based on the results in Table 1 and on testing of the process conditions and parameters it was concluded that excitation of tube 1 by a magnetic field of low frequency and of very low intensity has allowed the water nanostructures generated by the DNA fragment contained in this tube to be transmitted via waves to tube 2.

Step C:

Reconstitution by PCR of the LTR DNA from the Nanostructures in the Receiving or Signalized Sample.

A sample volume (5 μl) of tube 2-signalized water was added to 45 μl of an amplification mixture in a propylene 200 μl PCR tube (Eppendorf).

The amplification mixture was composed of (buffer composition) 0.2 mM dNTP's, 10 μM of each specific HIV-1 LTR primer containing the ingredients for synthesizing DNA, either from a positive dilution for EMS or in a lesser dilution, starting with 10⁻² down to 10⁻¹⁰: and using 1 unit of Taq DNA polymerase.

Once the first cDNA strand is synthesized, cycling of denaturation, annealing and polymerization steps are performed as usually used for the PCR amplification.

The reaction (35 cycles, T° annealing 56° C.) yielded a DNA band of the size (in electrophoresis migration in agarose 1.5%) of the expected 104 bp sequence. This amplicon was then cloned in a bacterial plasmid (Topo Cloning, Invitrogen) which was used to transform bacterial competent cells. Plasmid clones were purified from isolated bacterial transformants and screened for the presence of the 104 bp insert by EcoRI digestion. Positive plasmid clones are then sequenced and the sequence of the insert shown to be 98% to 100% identical (difference of 2 nucleotides) to the original DNA of tube 1.

The first step of DNA synthesis using the nanostructures as templates can also be achieved by a reverse transcriptase (RT) and other more classical DNA polymerase, at lower temperature (42° C. for example for the reverse transcription step). HIV1 LTR DNA D6 (EMS positive) dilution was used as emitter using excitation frequency of 7 Hz during 18 hours in the apparatus described in FIG. 1, and placed close to the water receiver Tube 2. Like the latter, it was then diluted at 10⁻², refiltered by 450 nM and 20 nM filters and diluted to 10⁻¹⁵.

Each dilution was then amplified by PCR for 35 cycles. Note the DNA bands detected at dilutions D2, (FD2), D3, D4, and D5. It has to be noted that the synthesis of the DNA LTR band is obtained in high water dilutions (up to 10⁻⁹) of the tube 2 containing the signalized water, indicating the transmission of the DNA information from tube to tube, in the presence of the ambient electromagnetic background. The same phenomenon was also observed in high dilutions of tube 1, indicating the synthesis of DNA at dilutions containing no DNA molecules.

This PCR technology can be applied to the detection of nanostructures in body fluid (plasma, urine) apparently devoid of the microorganisms from which they originate. In all cases, it is necessary to use mechanical agitation (vortex) at each water dilution in addition to the ambient or controlled electromagnetic background.

Table 2 shows the role of time of exposure to the 7 Hz frequency on EMS transmission from DNA to water. These results used the DNA LTR preparation as used for procedures reported in Table 1.

TABLE 2

| | Time of | | | Positive |
|---|---|---|---|---|
| Content | exposure (hr) | EMS | % over noise | dilutions |
| Control DNA tube | 2 | + | 57.3 | D4→ D8 |
| Water | 2 | − | 0 | |
| Water | 4 | − | 0 | |
| Water | 6 | − | 0 | |
| Water | 8 | ± | 6.4 | D4→ D8 |
| Water | 16 | + | 13.4 | D5→ D8 |
| Control DNA tube | 16 | + | 63 | D4→ D8 |

As shown above EMS were detected in the receiving sample after an exposure time of 8 or 16 hrs when the originating sample exhibited positive EMS at dilutions of D4 to D8 (10⁻⁴ to 10⁻⁸). No EMS was detected in water exposed for less than 8 hrs.

### EXAMPLE 2

### Identification of Unknown DNA Using Random Primers

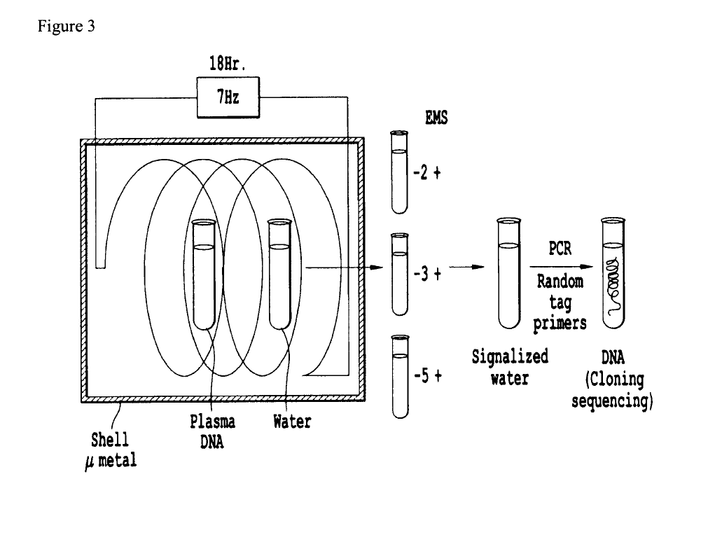

Another aspect of the invention is directed to a general procedure for the identification of any unknown DNA sequence (or polynucleotide sequence) capable of producing EMS in biological fluids. The principle is shown by FIG. 3. The transmission of EMS in water allows the selective transmission of only the DNA sequences that were emitting the EMS under the induction conditions. The PCR method uses a combination of random and Tag primers. The random primer associated with the Tag has the following formula 5′-GGACTGACGAATTCCAGTGACTNNNNNNNN (SEQ ID NO: 1) in which are made all possible combinations of 8 nucleotides for the 4 possible bases (65,536). A detailed procedure is described below.

1) DNA is purified from EDTA-collected human plasma extracted by the kit, QiaAMP, (Qiagen).

2) The purified DNA samples are filtered through 0.45 and 0.1 μm filters and then diluted to FD2-FD15 for analysis of EMS. FD2 refers to a filtered dilution of 1:100 or 10⁻².

3) The filtered and diluted samples are used to signalize water (molecular biology grade, 5Prime, 20 nm-filtered) with a dilution EMS+ of a patient DNA sample under an oscillating magnetic field of 7 Hz, 4V (coil in mu-metal) for 18 hours.

4) Each EMS+ sample used is filtered, vortexed and diluted (FD2-FD5) the signalized water sample and proceed to EMS analysis.

5) The samples of signalized water (EMS+), starting with FD2 are used as template for PCR amplification using random and Tag primers, following the protocol described below:

A 49 μl PCR amplification mix containing 1× Advanced Taq buffer with Mg²⁺ (available from 5Prime Co.), 200 μM dNTPs, 20 nM of designed random primer Tag8N (SEQ ID NO: 1):

(SEQ ID NO: 2)
(5′-GGACTGACGAATTCCAGTGACTNNNNNNNN)

20 μl of vortexed FD2 signalized water template, and 1 unit of Taq DNA polymerase (available from 5Prime Co.) is incubated stepwise at 8° C., 15° C., 20° C., 25° C., 30° C., 36° C., 42° C., and 46° C. for 2 min at each temperature to allow annealing of the random portion of the primer. An elongation step at 68° C. for 2-15 min was performed to allow synthesis of DNA, followed by a denaturation step at 95° C. for 3 min. One μl of the designed primer Tag-ONLY (5′-GGACTGACGAATTCCAGTGACT) (SEQ ID NO: 3) is added to the mixture at a final concentration of 200 nM. The resulting sample is subjected to 40 cycles of amplification (95° C./30 s, 59° C./30 s, and 70° C./2 min), followed by an incubation at 70° C. for 10 min. PCR-amplified samples are subjected to electrophoresis in 1.3% agarose gel and stained with ethidium bromide to allow visualization of amplified DNA bands under UV light.

6) If needed (if faint or no DNA bands a-re detected), sample can be reamplified by PCR using only the primer Tag-ONLY, following the reamplification protocol described below:

A 50 μl PCR amplification mix containing 1× Hot Start Taq buffer with Mg²⁺ (available from 5Prime Co.), 200 μM dNTPs, 200 nM of designed primer Tag-ONLY (5′-GGACTGACGAATTCCAGTGACT) (SEQ ID NO: 3), 1-10 μl of PCR-amplified sample as template, and 1 unit of Hot Taq DNA polymerase (available from 5Prime Co.) is denatured at 95° C. for 3 min and subjected to 25-40 cycles of amplification (95° C./30 s, 59° C./30 s, and 70° C./2 min), followed by an incubation at 70° C. for 10 min.

7) Isolation, purification and cloning of amplicons in pCR2.1-TOPO (InVitrogen) vector, followed by transformation of competent Escherichia coli cells, and screening for positive clones.

8) DNA sequencing of amplicons using M13 universal primers (Eurofins MWG GmbH, Germany) and BLAST of the resulting sequences.

Application to a patient suffering from chronic Lyme disease:

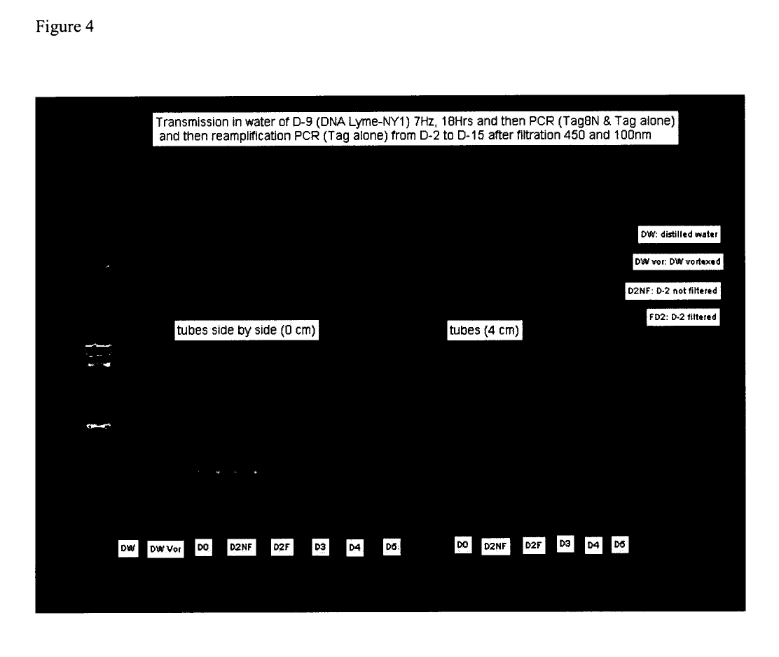

A D9 (10⁻⁹) dilution of DNA extracted from the plasma of a patient suffering from chronic Lyme disease was transduced into water at an excitation frequency of 7 Hz for 18 hrs. PCR was performed on the water sample after transduction with Tag8N primers followed by a second PCR with Tag primers only. The PCR DNA products were resolved on agarose gels by electrophoresis and are shown in FIG. 4. As shown at the left, results obtained when the tube of D9 DNA and the naive tube of water are placed side-by-side. At right, results obtained when the two tubes were placed at a distance of 4 cm from each other.

Dw denotes control, naïve, unsignalized water.

Dw vor: denotes control naïve, unsignalized water agitated with a vortex.

D0: water that was transduced but not diluted.

D2 NF: same as D0 but diluted by 1:100 (D2).

D2 same as D2 NF, but filtered.

D3, D4, D5 represent further serial dilutions of D2 to factors of 1:1,000 (D3); 1:10,000 (D4) and 1:100,000 (D5). All serial dilutions were vortexed between each 1:10 dilution.

### EXAMPLE 3

### Recording and Transduction of EMS Signatures of HIV and Borrelia Burgdorferi

EMS signatures of HIV DNA and Borrelia DNA sequences are recorded and transduced as described below.

Step 1: Preparation of DNAs

1. A fragment of HIV DNA taken from its long terminal repeat (LTR) sequence present in the viral DNA extracted from the plasma of a HIV-infected patient or obtained from a purified infectious DNA clone of HIV1 Lai, is amplified by PCR (487 base pairs) and nested PCR (104 base pairs) using specific primers: TR InS 5′-GCCTGTACTGGGTCTCT (SEQ ID NO: 4) and LTR InAS 5′-AAGCACTCAAGGCAAGCTTTA (SEQ ID NO: 5). A longer variant (300 bp) is obtained using the following primer: 5′-TGTTAGAGTGGAGGTTTGACA (SEQ ID NO: 6) in conjunction with the above primer InAS.

2. A DNA sequence from Borrelia Burgdorferi, the agent of Lyme disease, is amplified by PCR (907 base pairs) and nested PCR (499 base pairs) with respectively Borrelia 16S outer and inner primers. Inner BORR16S inS 5′-CAATCYGGACTGAGACCTGC (SEQ ID NO: 7) and BORR16S inAS 5′-ACGCTGTAAACGATGCACAC (SEQ ID NO: 8). A shorter variant of 395 bp is obtained by using the following primer: 5′-GACGTCATCCTCACCTTCCT (SEQ ID NO: 9) in conjunction with the above primer inAS.

Step 2: Signal Recording

The resulting amplicons 104 bp and 300 bp for LTR and 499 bp and 395 bp for Borrelia were prepared at a concentration of 2 ng/ml in a final volume of 1 ml of DNAse/RNAse-free distilled water. The samples were read on an electromagnetic coil, connected to a Sound Blaster card (Creative Labs) itself connected to a microcomputer, (preferably Sony VGN—CS31) preferentially powered by its 12 volt battery. Each emission is recorded for 6 seconds, amplified 500 times and the digital file is saved, for example under the form of a sound file with the .wav format. This file can later undergo digital processing, by a specific software, Matlab (Mathworks), as for example digital amplification for calibrating the signal level, filtering for eliminating unwanted frequencies, or be analyzed by transformation into its spectrum by a discrete Fourier transform, preferably by the algorithm of FFT “Fast Fourier Transform”.

Step 3: Signal Transduction in Water:

For transduction, the digital signal was converted by the digital/analog converter of the sound card into an analog signal. The output of the sound card of the microcomputer was linked to the input of a commercial amplifier (Kool Sound SX-250, www._conrad.com) having the following characteristics: passband from 10 Hz to 20 kHz, gain 1 to 20, input sensitivity 250 mV, output power RMS 140 W under 8 ohms.

The output of the amplifier was connected to a transducer solenoid which has the following characteristics: the bobbin has a length of 120 mm, an internal diameter of 25 mm, an external diameter of 28 mm, with 3 layers of 631 spirals of copper wire of 0.5 mm diameter and a resistance of 8 ohms, field on the axis to the centre 44 Oe/A, and on the edge 25 Oe/A. A measurement of 4.4 milliTesla (mT) was obtained when current, voltage and resistance were respectively, 100 mA, 4V and 8 ohms.

50 ml of DNAse/RNAse-free distilled water (5-Prime Ref 2500010) are filtered first through a sterile 450 nM filter (Millex, Millipore, Cat N° SLHV033RS) and then to a 20 nM filter (Whatman, Anotop 25, Cat N° 6809-2002). For transduction, 1 ml of this filtered water in a Eppendorf sterile tube of 1.5 ml was placed at the center of the solenoid, itself installed at room temperature on an isolated (non metal) working bench. Alternatively, a sterile tube of 15 ml (Falcon-Becton Dickinson), filled with the filtered water can be used instead of the 1.5 ml Eppendorf tube.

The modulated electric current produced by the amplifier was applied to the transducer coil for 1 hr at the tension of 4 Volts. A current intensity of 100 mA was applied to the coil, so that no significant heat was generated inside the cylinder.

Step 4: Reconstitution by PCR of the DNA from the Signalized Water.

The water which has received the recorded specific signal is called “signalized water”. The signalized water (kept in the same tube) was first agitated by strong vortex for 15 seconds at room temperature and then diluted 1/100 in non signalized DNAse/RNAse-free distilled water (30 μl/3 ml). 1 ml was kept for control (NF, nonfiltered), the 2 mls remaining of signalized water were filtered through a sterile 450 nM filter and then through a 100 nM (Millex, Millipore, Cat N° SLVV033RS) for Borrelia DNA or 20 nM filter (Whatman, notop25) for HIV DNA. The filtrate was then diluted serially 1 in 10 (0.1 ml in 0.9 ml of DNAse/RNAse-free distilled water) in a Eppendorf sterile tube of 1.5 ml from 10⁻² to 10⁻¹⁵ (D2 to D15). A strong vortex agitation was performed at each dilution step for 15 seconds. 5 μl of each dilution is added to 45 μl of the mix.

1. Preparation of the mix for HIV LTR: The PCR mixture (50 μl) contained 37.4 μl of DNAse/RNAse-Free distilled water, 5 μl of 10× Taq PCR buffer, 0.4 μl of 25 mM dNTPs, 1 μl of 50 μM each appropriate primer Inner [LTR InS (5′-GCCTGTACTGGGTCTCT) (SEQ ID NO: 10) and LTR InAS (5′-AAGCACTCAAGGCAAGCTTTA) (SEQ ID NO: 11)], 0.2 μl of 5 U/μl Taq DNA Polymerase and 5 μl of each dilution. The PCR was performed with the mastercycler ep (Eppendorf). The PCR mixtures were pre-heated at 68° C. for 3 min (elongation step), followed by 40 PCR cycles of amplification (95° C. for 30 s; 56° C. for 30 s; 70° C. for 30 sec). A final extension step was performed at 70° C. for 10 min.

2. Preparation of the mix for Borrelia: The PCR mixture (50 μl) contained 37.4 μl of DNAse/RNAse-Free distilled water, 5 μl of 10× Taq PCR buffer, 0.4 μl of 25 mM dNTPs, 1 μl of 50 μM each appropriate primer Inner [BORR16S inS (5′-CAATCYGGACTGAGACCTGC) (SEQ ID NO: 7) and BORR16S inAS (5′-ACGCTGTAAACGATGCACAC) (SEQ ID NO: 8)], 0.2 μl of 5 U/μl Taq DNA polymerase and 5 μl of each dilution. The PCR was performed with the mastercycler ep (Eppendorf). The PCR mixtures were pre-heated at 68° C. for 3 min (elongation step), followed by 40 PCR cycles of amplification (95° C. for 30 s; 61° C. for 30 s; 70° C. for 1 min). A final extension step was performed at 70° C. for 10 min.

Electrophoresis of the PCR products in 1.5% agarose gel: A band of 104 bp for HIV LTR and a band of 499 bp Borrelia DNA should be detected at several dilutions.

3. Sequencing: The DNA bands are cut and DNA is extracted using a Qiagen kit which also describes classical conditions for cloning in E. coli. The amplified specific DNA is then sequenced to show its identity to the original DNA.

### INCORPORATION BY REFERENCE

Each document, patent, patent application or patent publication cited by or referred to in this disclosure is incorporated by reference in its entirety, especially with respect to the specific subject matter surrounding the citation of the reference in the text or with regard to the pertinent portions of the invention supported by the reference. However, no admission is made that any such reference constitutes background art and the right to challenge the accuracy and pertinence of the cited documents is reserved.

1. A method for producing a sample that emits a detectable electromagnetic signal (EMS) signature for a self-replicating molecule comprising:
serially diluting a sample containing DNA or another self-replicating molecule in an aqueous solution or other diluent in which nanostructures characteristic of said molecule can be induced,
agitating the sample between serial dilutions, and
selecting a signalized sample determined to emit an EMS characteristic of said molecule.

2. The method of claim 1, wherein said self-replicating molecule is DNA and the serial dilution is made in water and the sample is agitated between serial dilutions by vortexing.

3. The method of claim 1, further comprising:
performing a signal analysis of the low frequency electromagnetic emission over time; and
producing an output, based on the signal analysis.

4. A method for transducing an EMS signature of a particular molecule into a naïve solution comprising:
placing an originating sample that emits a characteristic EMS signature next to a naïve sample inside of an electromagnetic coil that emits at a frequency of at least 7 Hz for a time and under conditions sufficient to transfer or transduce the EMS signature from the originating sample to the naïve sample thus producing an EMS signalized sample.

5. The method of claim 4, wherein the originating sample is DNA and the naïve sample is water.

6. The method of claim 4, further comprising contacting said EMS signalized sample with a primer or probe specific for a known nucleic acid sequence.

7. The method of claim 4, comprising detecting an electromagnetic signal characteristic of an HIV nucleic acid sequence (EMS signature) and comparing it to that detected from an otherwise identical sample not containing the HIV nucleic acid.

8. The method of claim 4, comprising detecting an electromagnetic signal characteristic of a Borellia nucleic acid sequence (EMS signature) and comparing it to that detected from an otherwise identical sample not containing the Borellia nucleic acid.

9. The method of claim 4, wherein said originating sample contains an unknown nucleic acid molecule and wherein said method further comprises contacting said EMS signalized sample with random primers, performing DNA amplification or PCR, and analyzing the PCR product to determine the sequence or other identifying characteristics of the unknown nucleic acid in the originating sample.

10. The method of claim 9 which comprises:
amplifying a nucleic acid in a test sample using random nucleotide sequence or polynucleotides or primers;
diluting and agitating during dilution the amplified nucleic acids in an aqueous solvent;
measuring over time a low frequency electromagnetic emission from the diluted amplified nucleic acids; and
optionally (i) identifying an EMS signature for amplified nucleic acid or its associated nanostructures by comparing the EMS of the test sample to the EMS of a control sample, and optionally (ii) comparing the results to relevant standard EMS signature(s).

11. The method of claim 4, wherein the originating sample is obtained from a subject having or suspected of having a parasitic or fungal disease or disorder.

12. The method of claim 4, wherein the originating sample is obtained from a subject having or suspected of having a bacterial disease or disorder.

13. The method of claim 4, wherein the test sample is obtained from a subject having or suspected of having a viral disease or disorder.

14. The method of claim 4, wherein the test sample is obtained from a subject having or suspected of having had an autoimmune disorder.

15. The method of claim 4, wherein the test sample is obtained from a subject having or suspected of having Alzheimer's Disease or Parkinson's Disease or any other neurological disease.

16. The method of claim 4, wherein the originating sample is obtained from a subject having a disease, disorder or condition of unknown or incomplete etiology in comparison with a noninfected subject.

17. A signalized sample produced by the method of claim 1.

18. An EMS signalized sample produced by the method of claim 4.

19. An amplified nucleic acid produced by the method of claim 9.

20. A device for producing an EMS signature in a solvent or an aqueous buffer comprising:
at least two containers, at least one for an EMS originating sample and at least one for an EMS receiving sample,
an electrically conducting coil that can emit a variable frequency ranging from 1 to 20,000 Hz, optionally connected to an external generator of alternating current having a variable frequency from 1 to 20,000 Hz,
means for electromagnetic shielding the at least two containers and the electrically conducting coil.
